(19) United States
(12) Patent Application Publication
(10) Pub. No.: US 2010/0191090 A1
(43) Pub. Date: Jul. 29, 2010

(54) MULTI-CHANNEL ELECTRODE SENSOR APPARATUS FOR SIMULTANEOUSLY MEASURING A PLURALITY OF PHYSIOLOGICAL SIGNALS

(75) Inventors: Seungchul Shin, Daejeon-city (JP); Young-Ju Jeon, Daejeon-city (KR); Seunghwan Kim, Daejeon-city (KR)

Correspondence Address: AMPACC Law Group, 3500 188th Street S.W., Suite 103, Lynnwood, WA, 98037, US

(73) Assignee: Electronics and Telecommunications Research Institute, Daejeon-city (KR)

(21) Appl. No.: 12/595,882

(22) PCT Filed: Jan. 25, 2008
(86) PCT No.: PCT/KR2008/000458

§ 371 Date: Oct. 14, 2009

(30) Foreign Application Priority Data
Apr. 30, 2007 (KR) 1020070042075

(51) Int. Cl. A61B 5/0416; A61B 5/04; A61B 5/0492

(52) U.S. Cl. 600/388; 600/393; 600/390

(57) ABSTRACT

A multi-channel electrode sensor apparatus for measuring a plurality of physiological signals is provided. The multi-channel electrode sensor apparatus includes a multi-channel electrode having a plurality of electrodes that are weaved using respective conductive yarns to measure potential differences of a plurality of physiological signals and an insulation fabric that is weaved using a non-conductive yarn to insulate the electrodes from each other and a snap connection unit that connects the multi-channel electrode to a measuring device to transmit the physiological signals to the measuring device.

### TECHNICAL FIELD

The present invention relates to a multi-channel electrode sensor apparatus for measuring a variety of physiological signals. More particularly, the present invention relates to an apparatus for simultaneously measuring a plurality of physiological signals, which appear as different electric potentials, using a multi-channel electrode sensor weaved using a conductive yarn.

This work was supported by the IT R&D program of MIC/IITA [2006-S-007-01, Ubiquitous Health Monitoring Module and System Development].

### BACKGROUND ART

A conductive electrode for contacting a human body is necessary when manufacturing belts or clothes for measuring a physiological signal. A disposable electrode, a dry electrode, an active dry electrode, a conductive patch electrode, a conductive rubber electrode, and the like are used as the conductive electrode.

Since the conductive electrode directly contacts skin, it is important to design the conductive electrode such that it does not cause skin problems. Therefore, in order to reduce skin problems and stably measure a physiological signal, the conductive electrode is weaved using a conductive textile coated with silver or other metals.

U.S. Pat. No. 6,970,731, entitled ‘Fabric-Based Sensors for Monitoring Vital Signs,’ filed with the USPTO on Nov. 14, 2000, by Georgia Tech Research Corp. and issued on Nov. 29, 2005 discloses a method of manufacturing a fabric electrode formed in a cloth or knit type using a conductive yarn and mounting the fabric electrode on clothes to measure physiological signals.

Next, U.S. Patent Publication No. 2006-0211934, entitled ‘Textile-Based Electrode,’ filed with the USPTO on Mar. 16, 2005 by Textronics, Inc. and published on Sep. 21, 2006, discloses a method of manufacturing a dual-structure electrode sensor having an outer fabric and an inner fabric and connecting a measuring device to the electrode sensor using a metal such as a snap to measure an electrocardiogram.

Further, U.S. Pat. No. 6,477,397, entitled ‘Electrode Structure,’ filed on May 18, 2000 with the USPTO by Polar Electro Oy and issued on Nov. 5, 2002, discloses a method of designing an electrode sensor using a conductive yarn as a filling yarn and using a nonconductive yarn as a warp yarn. The electrode sensor is weaved having peaks and valleys that are alternately structured to improve a contact property with the skin.

In addition, U.S. Pat. No. 6,272,365, entitled ‘Connecting Arrangement at Heart Rate Monitor and Electrode Belt,’ filed on Jun. 21, 1999, with the USPTO by Polar Electro Oy and issued on Aug. 7, 2001, discloses a method of connecting a heart rate measuring electrode belt to a surrounding band surrounding a human body using a socket type structure. According to this method, a heart rate of a person who is short and small can be easily measured.

According to the above-described prior art methods, since the electrodes are weaved by only one kind of conductive yarn, only a single channel signal can be measured. Therefore, there is a problem in that two or more channel physiological signals cannot be simultaneously measured. Furthermore, since the electrode is designed without considering shaking of the measuring device, slippage of the electrode from the belt or clothes by moisture such as sweat, or movement of a wearer, the physiological signals cannot be accurately measured when the wearer is exercising, e.g., running or practicing gymnastics.

### DISCLOSURE OF INVENTION

### Technical Problem

The present invention provides a multi-channel electrode sensor apparatus that is weaved using conductive yarns and thus configured to simultaneously measure a plurality of electric signals representing different physiological signals such as electro-cardiogram, breathing waver, electromyogram, body fat, and the like.

The present invention also provides a multi-channel electrode sensor apparatus having a support structure that is applied to various physiological signal measuring belts or clothes to stably measure a plurality of physiological signals even when a wearer is exercising, e.g., running or practicing gymnastics.

### Technical Solution

According to an aspect of the present invention, there is provided a multi-channel electrode sensor apparatus including a multi-channel electrode having a plurality of electrodes that are weaved using respective conductive yarns to measure potential differences of a variety of physiological signals and an insulation fabric that is weaved using a non-conductive yarn to insulate the electrodes from each other; and a snap connection unit that connects the multi-channel electrode to a measuring device to transmit the physiological signals to the measuring device.

According to another aspect of the present invention, there is provided a multi-channel electrode sensor apparatus including a multi-channel electrode having one or more electrodes that are weaved using respective conductive yarns to measure potential differences of a variety of physiological signals and an insulation fabric that is weaved using a non-conductive yarn to insulate the electrodes from each other, and one or more metallic snap connection units that connect the multi-channel electrode to a physiological signal measuring device; a support maintaining a close contact state between the multi-channel electrode and the metallic snap connection units and making the attaching/detaching of the physiological signal measuring device easy; and a slippage preventing unit for preventing slippage of the multi-channel electrode on a contacting portion of a human body.

### Advantageous Effects

According to the present invention, the multi-channel electrode sensor apparatus for measuring a plurality of physiological signals can simultaneously measure a variety of electric signals representing different physiological signals such as electrocardiogram, breathing waver, electromyogram, body fat, and the like in a state where it is mounted on a belt or clothing. In addition, the metallic snap and the slippage preventing unit functions as an electric signal transmission passage as well as a support for preventing the measuring device from shaking. Therefore, the slippage of the electrode from a portion contacting with a human body by moisture such as sweat or rain, or movement of a wearer can be prevented. Furthermore, since the measuring device can be fixed by the connection snap without using other mechanical structures, the structure of the apparatus can be simplified and the physiological signals can be accurately measured even when the wearer is exercising, e.g., running or practicing gymnastics. In addition, the physiological signals can be compared by measuring identical types of physiological signals at more than two channels.

### DESCRIPTION OF DRAWINGS

The above and other features and advantages of the present invention will become more apparent by describing in detail exemplary embodiments thereof with reference to the attached drawings in which:

FIG. 1 is a schematic diagram of a multi-channel electrode sensor apparatus for measuring a plurality of physiological signals according to an embodiment of the present invention;

FIG. 2 is a front view of a multi-channel electrode sensor apparatus for measuring a plurality of physiological signals according to an embodiment of the present invention;

FIG. 3A is a rear view of a multi-channel electrode sensor apparatus according to an embodiment of the present invention.

FIG. 3B is a sectional view taken along line A-A′ of FIG. 2;

FIG. 4 is a rear view of a multi-channel electrode sensor apparatus according to another embodiment of the present invention.

FIG. 5 is a rear view of a multi-channel electrode sensor apparatus according to another embodiment of the present invention.

FIG. 6 is a perspective view of a chest belt on which a multi-channel electrode sensor apparatus for measuring a variety of physiological signals according to an embodiment of the present invention is installed; and

FIG. 7 is a perspective view of a clothing on which a multi-channel electrode sensor apparatus for measuring a variety of physiological signals according to an embodiment of the present invention is installed.

### BEST MODE

The attached drawings for illustrating preferred embodiments of the present invention are referred to in order to gain a sufficient understanding of the present invention, the merits thereof, and the objectives accomplished by the implementation of the present invention.

Hereinafter, the present invention will be described in detail by explaining preferred embodiments of the invention with reference to the attached drawings. Like reference numerals in the drawings denote like elements.

FIG. 1 is a schematic diagram of a multi-channel electrode sensor apparatus for measuring a variety of physiological signals according to an embodiment of the present invention.

Referring to FIG. 1, a multi-channel electrode sensor apparatus 100 includes a multi-channel electrode 110 having a plurality of electrodes 111, 112, and 113 that are weaved using respective conductive yarns to measure potential differences of a plurality of physiological signals and an insulation fabric 114 that is weaved using a non-conductive yarn to insulate the electrodes 111, 112, and 113 from each other, and a snap connection unit 120 that connects the multi-channel electrode 110 to a measuring device to transmit the physiological signals to the measuring device. The snap connection unit 120 includes a plurality of metal connection members that respectively connect the electrodes 111, 112, and 113 to the measuring device.

The physiological signals are signals representing at least one of electrocardiogram, breathing waver, electromyogram, body fat, and body resistance. In this embodiment, the physiological signals are simultaneously measured.

The multi-channel electrode sensor apparatus 100 further includes a support allowing the multi-channel electrode 110 and the snap connection unit 120 to closely contact each other, a slippage preventing unit that is formed of fabric, rubber, or the like to fix the multi-channel electrode 110 on a body portion, and an elastic band for connecting the multi-channel electrode sensor apparatus 100 to a physiological measuring belt or clothing.

According to a feature of this embodiment, the electrodes 111, 112, and 113 are weaved using respective conductive yarns having different electric conductivities or made having different contacting areas with a human body for measuring various physiological signals.

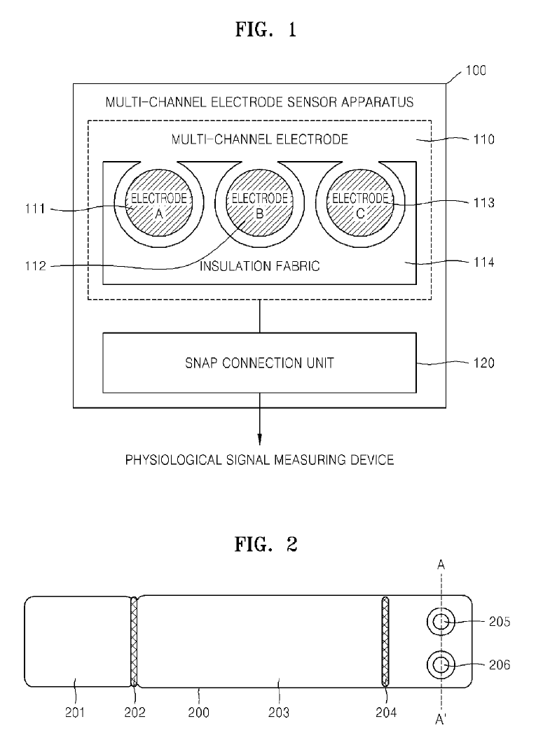

FIG. 2 is a front view of a multi-channel electrode sensor apparatus for measuring a plurality of physiological signals according to an embodiment of the present invention.

FIG. 2 illustrates the multi-channel electrode sensor apparatus of FIG. 1 in detail. Referring to FIG. 2, the multi-channel electrode sensor apparatus includes a multi-channel electrode sensor that is weaved using an conductive yarn to measure the potential differential, an elastic band 201 for connecting the multi-channel electrode sensor 200 to a belt or a clothing, a slippage preventing unit 203 that is formed of rubber or fabric, a sewing line 202 for connecting the slippage preventing unit 203 to the elastic band 201, a sewing line 204 for fixing the support, a first snap connection unit 205 for connecting the measuring device to the electrode sensor 200, and a second snap connection unit 206 for connecting the measuring device to the electrode sensor 200.

In order to describe the multi-channel electrode sensor apparatus in more detail, rear and side views of the apparatus and several modified examples of the multi-channel electrode sensor apparatus will be described.

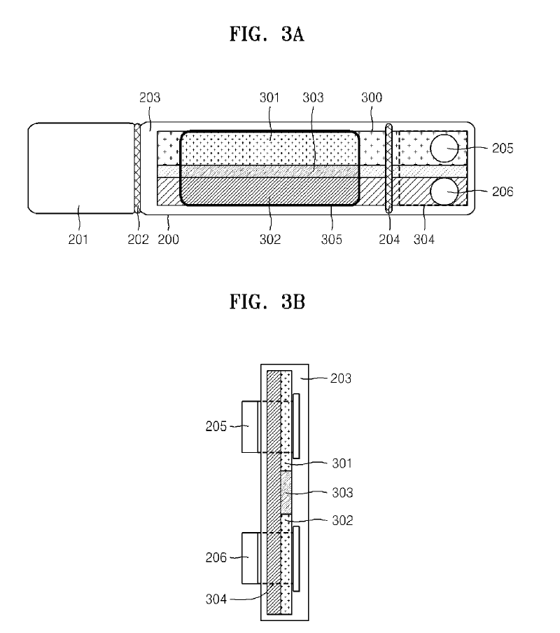

FIG. 3A is a rear view of the multi-channel electrode sensor apparatus of FIG. 2.

Referring to FIG. 3A, a multi-channel electrode 300 includes first and second electrodes 301 and 302 weaved using conductive yarns and an insulation fabric 303 for insulating the electrodes 301 and 302 from each other. Here, the multi-channel electrode 300 is connected to a measuring device through first and second snap connection units 205 and 206 in order to transmit and receive electric signals.

A support 304 for securely fixing the snap connection units 205 and 206 to the multi-channel electrode 300 is provided. The support 304 is formed of a hard material such as plastic or polymer. The support 304 is fixed by a sewing line 204 which prevents the support 304 from being shaken. The multi-channel electrode 300 is surrounded by a slippage preventing unit 203 formed of fabric or rubber. Particularly, a portion around a surface 305 contacting with a skin is specially sewed.

FIG. 3B is a sectional view taken along line A-A′ of FIG. 2.

Referring to FIG. 3B, the first and second electrodes 301 and 302 that are weaved using the respective conductive yarns are insulated from each other by the insulation fabric 303 weaved using the non-conductive yarn. The snap connection units 205 and 206 connected to the respective electrodes 301 and 302 are designed to penetrate the electrodes 301 and 302 and the support 304.

The snap connection units 205 and 206 are provided to transmit the physiological signals detected by the electrode sensor to the measuring device by contacting the measuring device. Therefore, the snap connection units 205 and 206 are formed of a conductive metal. By providing more than two snap connection units 205 and 206, a contact error that may be caused by shaking between the measuring device and the snap connection units 205 and 206 can be reduced.

### MODE FOR INVENTION

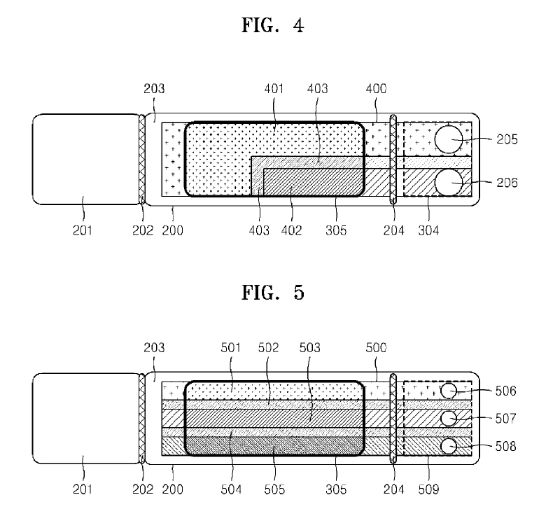

FIG. 4 is a rear view of a multi-channel electrode sensor apparatus according to another embodiment of the present invention.

Referring to FIG. 4, a multi-channel electrode 400 includes first and second electrodes 401 and 402 that are weaved using conductive yarns having identical or different conductivities and an insulation fabric 403 for insulating the electrodes 401 and 402 from each other. The multi-channel electrode sensor measures the signals by making contact areas of the electrodes to be different to the human body or by making the electrodes to have different conductivities.

Particularly, since the contact areas of the electrodes are different from each other, a variety of physiological signals can be measured.

FIG. 5 is a rear view of a multi-channel electrode sensor apparatus according to another embodiment of the present invention.

Referring to FIG. 5, a multi-channel electrode sensor apparatus of this embodiment has three electrodes. That is, a multi-channel electrode 500 includes electrodes 501, 502, and 503 that are weaved using different conductive yarns and insulation fabrics 502 and 504 for insulating the electrodes 501, 502, and 503 from each other. The electrodes 501, 503, and 505 are respectively fixed by metallic snap connection units 506, 507, and 508 penetrating a support 509. Here, the snap connection units 506, 507, and 508 are respectively connected to snaps installed on the measuring device. If the snap connection units 506, 507, and 508 have protruding type metallic snaps, snaps installed on the measuring device are groove type metallic snaps in which the protruding type snaps are fitted.

As described above, since the electrode sensor includes a plurality of electrodes, a plurality of physiological signals can be measured. In addition, as described above, the plurality of physiological signals can be also measured by making the contacting areas of the electrodes different.

The electrodes may be weaved using identical conductive yarns which have the same conductivity. Alternatively, the electrodes may be weaved using different conductive yarns having different conductivities considering properties of signals that will be measured by the respective electrodes. Therefore, various types of multi-channel electrode sensors can be implemented.

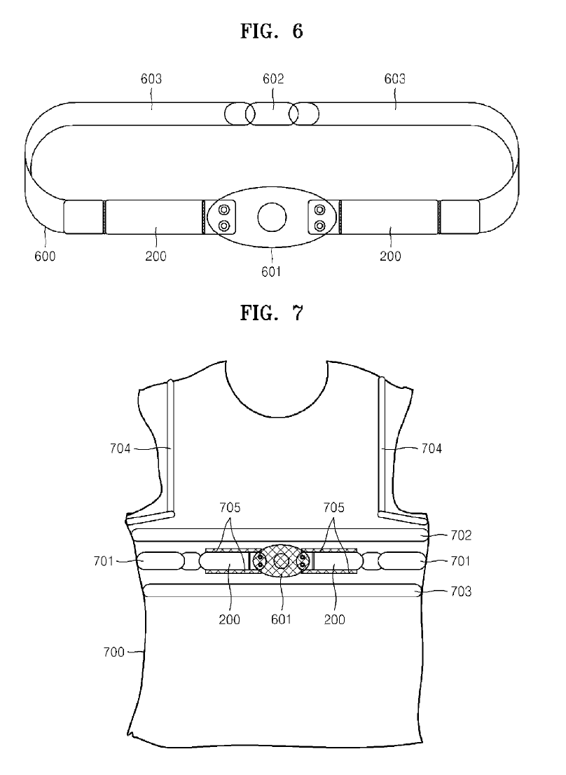

FIG. 6 is a perspective view of a chest belt on which a multi-channel electrode sensor apparatus for measuring a variety of physiological signals according to an embodiment of the present invention is installed.

FIG. 6 illustrates a chest belt 600 formed using the multi-channel electrode sensor 200 and connected to a physiological signal measuring device 601. Referring to FIG. 6, the chest belt 600 includes the multi-channel electrode sensors 200 and elastic bands 603 connected to the multi-channel electrode sensors 200. The chest belt 600 is worn around the chest with a proper tightness using a band length adjusting unit 602.

The physiological signal measuring device 601 and the chest belt 600 are fixed by more than two snap connection units to simultaneously measure different physiological signals such as electrocardiogram, breathing waver, electromyogram, body fat, and the like. Here, the snap connection units function as electric signal transmission passages as well as fixing unit for preventing the measuring device 601 from shaking on the chest belt 600. As a result, there is no need for a special unit for preventing the measuring unit 601 from shaking. The elastic bands 603 may be formed by integrally extending from the multi-channel electrode sensors 200 or by connecting different fabrics through needlework.

FIG. 7 is a perspective view of a clothing on which a multi-channel electrode sensor apparatus for measuring a variety of physiological signals according to an embodiment of the present invention is installed.

FIG. 7 illustrates a clothing 700 formed using the multi-channel electrode sensor 200 and connected to a physiological signal measuring device 601. Referring to FIG. 7, the multi-channel electrode sensor 200 is attached to the clothing 700 through needlework for contacting with a human body and a tightening unit 701 such as a Velcro-fastener is used to allow the electrode sensor 200 to closely contact the human body.

The clothing 700 includes an upper dividing line 702 for reducing affection of an upper body motion on a signal measurement, a lower dividing line 703 for reducing affection of a abdomen motion, and left and right dividing lines 704 for reducing left and right arms motion. The dividing lines may be formed of fabrics having very good elasticity such as spandex or a mesh type fabric.

The invention can also be embodied as computer readable codes on a computer readable recording medium. The computer readable recording medium is any data storage device that can store data which can be thereafter read by a computer system. Examples of the computer readable recording medium include read-only memory (ROM), random-access memory (RAM), CD-ROMs, magnetic tapes, floppy disks, optical data storage devices, and carrier waves (such as data transmission through the Internet). The computer readable recording medium can also be distributed over network coupled computer systems so that the computer readable code is stored and executed in a distributed fashion.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A multi-channel electrode sensor apparatus comprising:
a multi-channel electrode having a plurality of electrodes that are weaved using respective conductive yarns to measure potential differences of a plurality of physiological signals and an insulation fabric that is weaved using a non-conductive yarn to insulate the electrodes from each other; and
a snap connection unit that connects the multi-channel electrode to a physiological signal measuring device to transmit the physiological signals to the measuring device.

2. The multi-channel electrode sensor apparatus of claim 1, further comprising:
a support maintaining a close contact state between the multi-channel electrode and the snap connection unit; and
a slippage preventing unit for preventing slippage of the multi-channel electrode on a contacting portion of the human body.

3. The multi-channel electrode sensor apparatus of claim 1, further comprising an elastic band connecting the multi-channel electrode sensor apparatus to a physiological signal measuring belt or clothing.

4. The multi-channel electrode sensor apparatus of claim 1, the snap connection unit includes metal connection members for respectively connecting the electrodes of the multi-channel electrode to the measuring device to transmit the physiological signals to the measuring device.

5. The multi-channel electrode sensor apparatus of claim 1, wherein the physiological signals are signals representing at least one of electrocardiogram, breathing waver, electromyogram, body fat, and body resistance and simultaneously measured.

6. The multi-channel electrode sensor apparatus of claim 1, wherein the electrodes of the multi-channel electrode are weaved using conductive yarns having different conductivities.

7. The multi-channel electrode sensor apparatus of claim 1, wherein the electrodes of the multi-channel electrode have different body contact areas to measure the respective physiological signals.
